(19) United States
(12) Patent Application Publication
(10) Pub. No.: US 2026/0346477 A1
(43) Pub. Date: Nov. 5, 2026

(54) END-EFFECTOR FOR ROBOTIC SYSTEMS

(71) Applicant: MULTI-SCALE MEDICAL ROBOTICS CENTER LIMITED, Hong Kong (CN)

(72) Inventors: Ka Chun LAU, Hong Kong (CN); Shi Pan SIU, Hong Kong (CN); Yeung YAM, Hong Kong (CN); Wai Yan Philip CHIU, Hong Kong (CN)

(21) Appl. No.: 19/120,767

(22) PCT Filed: Oct. 12, 2023
(86) PCT No.: PCT/IB2023/060259

§ 371 Date: Apr. 12, 2025

Related U.S. Application Data
(60) Provisional application No. 63/415,631, filed on Oct. 12, 2022.

(51) Int. Cl. B25J 15/00; A61B 34/30; B25J 15/12

(52) U.S. Cl. CPC B25J 15/0028; A61B 34/30; B25J 15/12; A61B 2034/303

(57) ABSTRACT

This invention provides an end-effector for robotic systems. In one embodiment, said end-effector for robotic systems, comprises: a) a first member; b) a second member comprising: i) two compliant structures, each comprising a living hinge; ii) a rigid bottom beam for connection with a tendon; iii)a pivot point in contact with said first member; wherein said second member is housed within said first member and pulling of said tendon actuates said second member to move at said pivot point and said two compliant structures to bend at said living hinge.

### FIELD OF THE INVENTION

The present invention generally relates to end-effectors for robotic systems, particularly a compliant gripper for robotic manipulators.

### BACKGROUND OF THE INVENTION

Gastric and colorectal cancers were commonly found around the world. They are also the top leading causes of cancer death worldwide. Practically, minimally invasive surgery is widely used to evaluate illnesses and injuries which does not need a large incision during surgery. Wherein, Endoscopic Submucosal Dissection (ESD) and Endoscopic Mucosal Resection (EMR) were well-developed to remove pre-malignant and early cancers in the GI tract. These procedures are performed with a flexible endoscope which allows the patient to recuperate faster with less pain.

With the rapid recent development of the endoscopic surgical platforms, the development of articulated end-effectors for this kind of novel handheld robotic tool is becoming even more important for this emerging field of medical robotics. As for the configuration of the current handheld design, it is hard to handle the resection in the fundus of the stomach because the enclosure of the end-effector impedes the retroreflective (J-shaped bending) of the over-tube. In addition, due to the characteristics of the working environment, there are significant limitations in the geometry and size of the end effectors. Developing reliable endoscopic manipulators has been a long challenge above barriers.

The compliant gripper with superior mechanical properties including a compliant beam of the grasping jaws which is an elastic mechanism that provides flexibility necessary on undergoing elastic deformation. This design can manipulate its X-Y plane and grasp the target object precisely. In addition, the passively compliant underactuated mechanism is one of the ways which allow the gripper to accommodate irregular objects with any shape and surface properties. Due to the jaws being capable of grasping delicate objects without damage to tissue which enhanced the safety during the surgery.

The purpose of the underactuated is used to optimize the gripper mechanism to control more degrees of freedom with less active input when driving the open and close motion of the gripper. Another purpose of underactuated is used to simplify the algorithm by reducing control variables. Apart from reducing the complexity of control required, the change also reduces the stiffness of the robot arm when bending.

### SUMMARY OF THE INVENTION

This invention provides an end-effector for a robotic system. In one embodiment, said end-effector for a robotic system, comprises: a) a first member; b) a second member comprising: i) two compliant structures, each comprising a living hinge; ii) a rigid bottom beam for connection with a tendon; iii)a pivot point in contact with said first member; wherein said second member is housed within said first member and pulling of said tendon actuates said second member to move at said pivot point and said two compliant structures to bend at said living hinge.

### BRIEF DESCRIPTION OF THE DRAWINGS

Exemplary non-limiting embodiments of the invention are described below with reference to the attached drawings. The drawings are illustrative and generally not to an exact scale. The same or similar elements on different figures are referenced with the same reference numbers.

FIGS. 1A to 1G shows various embodiments of the gripper structure for robotic manipulator.

### DETAILED DESCRIPTION OF THE INVENTION

This invention relates to end-effector for robotic system to accomplish objects gripping through implementation of compliant mechanisms.

Described herein is the structure of complaint gripper comprises living hinges to be used as an end-effector in robotic system. The structure is normally close so that the stiffness of the robotic arm and over-tube are not affected when the gripper is grasping objects. The opening motion is controllable through adjusting the pulling force of the tendon(s).

In one embodiment, the present invention provides a gripper structure for robotic manipulator, said gripper structure includes an opened end of a cylinder housing and a compliant gripper. The jaws are appended to a compliant beam that can safely grasp the tissue. This device can attach to a flexible robot arm. This device is underactuated which reduces the number of tendons on the actuation of the end effector and reduces the stiffness during bending.

This invention provides a structure for gripper to be used in robotic system. In one embodiment, said structure comprises: a first member which is cylindrical, and functions as a platform for the second members to mount on; two second members in which each of the members consists of at least one compliant segment that undergo elastic deformation during the operation of gripper; a rigid segment that is connected to each of the compliant segments which does not deform intentionally during the operation of gripper; wherein the gripper is normally closed; wherein the gripper is opened through pulling of at least one wire attached to the rigid segment.

In one embodiment, said structure further comprises at least one revolute joint interconnecting the complaint segment and the rigid segment.

In one embodiment, said structure further comprises an anchor for fixing the wire to the rigid segment.

In one embodiment, the opening angle of gripper is proportional to the pulling force applied on the wire.

In one embodiment, said structure further comprises a pivot on the cylindrical member that is coincident with the revolute joint of the complaint segments.

In one embodiment, said structure further comprises a metal piece that passes through the pivot and revolute joint for keeping the members in place.

In one embodiment, the first and second members are plastic.

In one embodiment, the second members are rhombus-shaped.

In one embodiment, each compliant segments the length-to-width ratio and the length-to-thickness ratio are larger than 1, and the thickness of the bottom rigid segment is larger than the thickness of compliant segment.

In one embodiment, the first member is the open-ended cylinder and comprises two identical tongues on the flat surface.

In one embodiment, said structure further comprises the lead-in feature which is in contact with groove of the bottom rigid segment to limit the rotation angle.

In one embodiment, the compliant segment comprises the living hinge.

In one embodiment, the rigid segment comprises grove.

This invention provides an end-effector for robotic systems. In one embodiment, said end-effector for robotic systems, comprises: a) a first member; b) a second member comprising: i) two compliant structures, each comprising a living hinge; ii) a rigid bottom beam for connection with a tendon; iii)a pivot point in contact with said first member; wherein said second member is housed within said first member and pulling of said tendon actuates said second member to move at said pivot point and said two compliant structures to bend at said living hinge.

In one embodiment, said first member comprises a lead in feature (110) in contact with a groove (109) at said rigid bottom beam (105) to limit rotation angle.

In one embodiment, said second member comprises: a) a first jaw comprising one of said two compliant structures, said rigid bottom beam and a first revolute joint; b) a second jaw attached to said first jaw, comprising one of said two compliant structures and a second revolute joint; wherein said first revolute joint and second revolute joint are joined to form said pivot point.

In one embodiment, said first member comprises a hole for insertion of a pin through said second member to form said pivot point.

In one embodiment, each of said two compliant structures comprises a compliant beam (104) and a top compliant segment (107).

In one embodiment, said second member is rhombus shaped wherein: a) said top compliant segment is hairpin shaped and comprises two ends; wherein said top compliant segment connects to said pivot point at one of said two ends and said compliant beam at another said two ends; b) said compliant beam comprises two ends; wherein said compliant beam connects said rigid bottom beam at one of said two ends and said top compliant segment at another said two ends.

In one embodiment, said first member is an open-ended cylinder with two identical tongues.

In one embodiment, said living hinge is curved.

In one embodiment, said tendon is connected at said rigid bottom beam with a hollow metal piece and passed through said first member.

In one embodiment, each compliant structure comprises a length-to-width ratio and a length-to-thickness ratio larger than 1 and said rigid bottom beam comprises a thickness larger than each compliant structure.

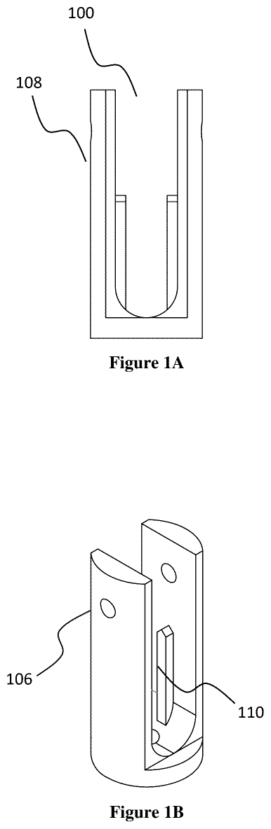

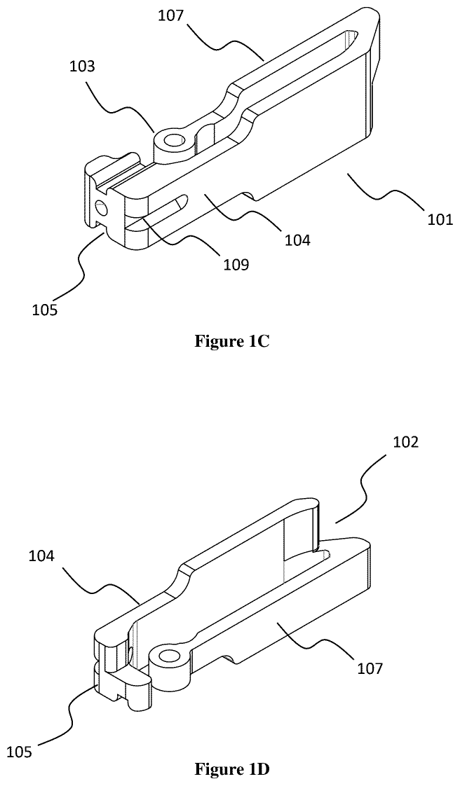

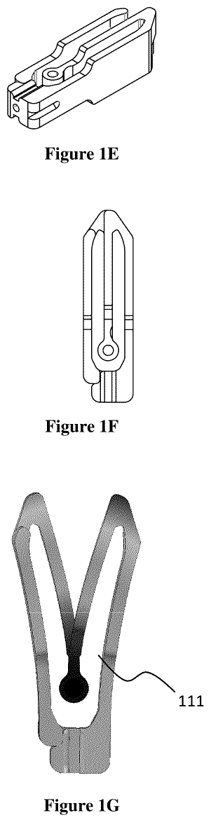

This invention provides a gripper structure for robotic manipulator. In one embodiment, said gripper structure for robotic manipulator is a gripping or a retracting device for robotic endoscopic surgery, comprising: A first member has an opening of a cylinder housing with a hollow 100 as depicted in FIG. 1A. A second member has two separable compliant structures 101,102, a gripper segment interconnecting the top and middle revolute joint 103, a compliant beam 104 interconnecting the rigid bottom beam 105. FIG. 1C shows the right jaw which is attracted to the compliant beam. FIG. 1D shows the left jaw which is attracted to the compliant beam and positioned to lie in confronting relation to the right jaw. Three individual parts are assembled by a metal pin, wherein the pivot point 106 of the second member is located on the first member. A tendon is in contact with an anchor to fix on the bottom beam 105, and pulling force is applied to the bottom rigid beam, wherein the first member is arranged for opening and closing motion in response to the force applied through the tendons, bottom rigid beam 105 translated rearwardly along the guiding of the first member in contact.

In one embodiment, the first member and second are made of a plastic material.

In one embodiment, the second member is rhombus-shaped and includes a top compliant segment 107 interconnected to the middle revolute joint 103, the top compliant segment coupled to the bottom rigid beam, each compliant segment has a width and a relatively smaller thickness which according to its material's properties, and the thickness of the bottom rigid beam is larger than the compliant segment.

In one embodiment, the first member is the open-ended cylinder and comprises two identical tongues 108 on the flat surface.

In one embodiment, the tendon is anchored at the bottom rigid beam 105 with hollow metal piece and passed through the circular housing.

In one embodiment, the compliant beam comprises the living hinge 111.

In one embodiment, the rigid beam comprises grove 109 which is a curved surface.

In one embodiment, the revolute joint is a torus.

In one embodiment, the living hinge 111 is curved.

In one embodiment, the tongue comprises the lead-in feature 110 which is in contact with groove of the bottom rigid segment to limit the rotation angle.

What is claimed is:

1. An end-effector for robotic systems, comprising:
a. a first member;
b. a second member comprising:
i. two compliant structures, each comprising a living hinge;
ii. a rigid bottom beam for connection with a tendon;
iii. a pivot point in contact with said first member;
wherein said second member is housed within said first member and pulling of said tendon actuates said second member to move at said pivot point and said two compliant structures to bend at said living hinge.

2. The end-effector of claim 1, wherein said first member comprises a lead in feature (110) in contact with a groove (109) at said rigid bottom beam (105) to limit rotation angle.

3. The end-effector of claim 1, wherein said second member comprises:
a. a first jaw comprising one of said two compliant structures, said rigid bottom beam and a first revolute joint;
b. a second jaw attached to said first jaw, comprising one of said two compliant structures and a second revolute joint;
wherein said first revolute joint and second revolute joint are joined to form said pivot point.

4. The end-effector of claim 1, wherein said first member comprises a hole for insertion of a pin through said second member to form said pivot point.

5. The end-effector of claim 1, wherein each of said two compliant structures comprises a compliant beam (104) and a top compliant segment (107).

6. The end-effector of claim 5, wherein said second member is rhombus shaped wherein:
a. said top compliant segment is hairpin shaped and comprises two ends; wherein said top compliant segment connects to said pivot point at one of said two ends and said compliant beam at another said two ends;
b. said compliant beam comprises two ends; wherein said compliant beam connects said rigid bottom beam at one of said two ends and said top compliant segment at another said two ends.

7. The end-effector of claim 1, wherein said first member is an open-ended cylinder with two identical tongues.

8. The end-effector of claim 1, wherein said living hinge is curved.

9. The end-effector of claim 1, wherein said tendon is connected at said rigid bottom beam with a hollow metal piece and passed through said first member.

10. The end-effector of claim 1, wherein each compliant structure comprises a length-to-width ratio and a length-to-thickness ratio larger than 1 and said rigid bottom beam comprises a thickness larger than each compliant structure.
